(19) United States
(12) Patent Application Publication
(10) Pub. No.: US 2003/0129161 A1
(43) Pub. Date: Jul. 10, 2003

(54) Interleukin-12 as a veterinary vaccine adjuvant

(76) Inventor: Hsien-Jue Chu, Fort Dodge, IA (US)

Correspondence Address: Anne M. Rosenblum, Esq., Suite 212, 163 Delaware Avenue, Delmar, NY, 12054, US

(21) Appl. No.: 10/243,075

(22) Filed: Sep. 12, 2002

Related U.S. Application Data
(60) Provisional application No. 60/322,840, filed on Sep. 17, 2001.

(51) Int. Cl. A61K 38/20; A61K 39/00

(52) U.S. Cl. 424/85.2; 424/184.1

(57) ABSTRACT

This disclosure describes a composition for enhancing the immunogenicity of a veterinary vaccine that comprises a pharmacologically effective amount of an immunomodulator and an immunoadjuvant. Additionally, the disclosure describes a vaccine composition comprising an effective immunizing amount of an antigen, an immunomodulator, an immunoadjuvant and a pharmaceutically acceptable carrier. The compositions may optionally contain conventional, secondary adjuvants or preservatives. The disclosure further describes a unique method for enhancing or accelerating the immunogenicity of weak, immunosuppressive or marginally safe antigens by administering to an avian or mammalian species a pharmacologically effective amount of the aforesaid immunogenicity enhancing composition or an effective immunizing amount of the aforesaid vaccine composition.

### CROSS-REFERENCE TO RELATED U.S. APPLICATIONS

This application claims the benefit under 35 U.S.C. §119 (e) of U.S. Provisional Application No. 60/322,840, filed Sep. 17, 2001. The prior application is incorporated herein by reference in its entirety.

### STATEMENT REGARDING FEDERALLY SPONSORED RESEARCH OR DEVELOPMENT

Not Applicable

### REFERENCE TO A “SEQUENCE LISTING”

Not Applicable

### BACKGROUND OF THE INVENTION

1. Field of the Invention

The present invention concerns a novel combination comprising an immunomodulator in conjunction with immunoadjuvants that enhances the immunogenicity or physiological efficacy of veterinary vaccines containing an antigen and the new use of the combination to significantly improve the immunological response of an animal to the antigen when administered concurrently or in admixture with a vaccine composition.

2. Description of the Related Art

All patents and publications cited in this specification are hereby incorporated by reference in their entirety.

The etiology of many debilitating or fatal diseases has been established. For example, Bovine Respiratory Syncytial Virus (hereinafter referred to as “BRSV”) is recognized as a significant factor in Bovine Respiratory Disease Complex. The disease is characterized by rapid breathing, coughing, loss of appetite, ocular and nasal discharge as well as elevated temperatures in cattle. Death can occur within 48 hours after onset of symptoms in an acute outbreak. BRSV is considered the most common viral pathogen in enzootic pneumonia in calves, and has also been associated with pulmonary emphysema among newly weaned calves.

Another disease of large animals, Strangles, is caused by a bacterial infection of Streptococcus equi. Also known as distemper or barn fever, Strangles is a highly contagious disease of a horse's upper respiratory tract characterized by the presence of local and disseminated abscesses.

A variety of etiologic agents are known to cause disease in small animals. Disease in dogs, for instance, is found to be associated with the presence of Ehrlichia canis, canine parvovirus (CPV), canine parainfluenza virus (CPI), canine adenovirus type II (CAV-2), canine adenovirus (CDV), canine coronavirus (CCV), Leptospira icterohemorrhagiae (LI), Leptospira canicola (LC), Leptospira grippotyphosa (LG), Leptospira pomona (LP) and the like. Similarly, disease in cats is caused by transmittable viruses such as feline immunodeficiency virus and feline leukemia virus among others, bacteria such as feline Chlamydia psittaci, etc.

There is a real need for effective prophylaxis against these types of etiologic agents that produce highly contagious, debilitating and deadly diseases in animals. However, veterinary vaccines often suffer from poor immunogenicity responses due to weak antigenic activities of certain etiologic agents or due to biological variations from one animal species to another. Reduced physiological efficacy is also problematic in any attempt to obtain proper humoral immune responses in animals. Producing an adequate level of serum antibodies, which reflect true protection against the disease through concomitant cell-mediated immunity, is difficult to achieve. Moreover, physical and chemical compatibilities of the antigenic substances with each additive or combination of additives must be resolved through significant testing to preclude rendering sensitive antigens inactive. Troublesome side effects or potential toxicity from a narrow margin of safety provide yet another challenge to the development of a useful veterinary vaccination program. Establishing protective immunity is not a simple matter. Thus, research has focused on finding a reliable, nontoxic adjuvant that is compatible with the antigen and able to improve the immunogenicity and efficacy of animal vaccines without raising toxicity concerns.

A number of immunoadjuvants has been examined and many hold promising abilities to augment cell-mediated and humoral immune responses to a variety of antigens suffering from weak immunogenicity (see discussion in R. Rabinovich, “Vaccine Technologies: View to the Future,” Science 265:1401-1404 (Sep. 2, 1994) and F. Audibert, “Adjuvants: current status, clinical perspectives and future prospects,” Immunology Today 14(6):281-284 (1993)). Alum (aluminum potassium sulfate), found in diphtheria, tetanus and hepatitis B vaccines, stimulates the humoral immune response but not the cell-mediated immunity. As a result, the salt is not efficacious with all immunogens. The aluminum salts also have the disadvantage of not lending themselves or the vaccines to lyophilization or freezing. Due to the limitations of the aluminum salts, research has turned to many alternative immunoadjuvants such as saponins, non-ionic block polymer surfactants, monophosphoryl lipid A, muramyl dipeptides (squalene oil) or tripeptides and cytokines. However, the selection of a suitable immunoadjuvant system is not an easy matter and requires substantial experimentation to discover if the system will enhance cell-mediated and humoral immune responses in a particular species of animal to different immunogens. Maintaining the stability and the efficacy of the immunogens are other important factors that can influence the selection process in finding whether the immunoadjuvant system will function as desired in the animal.

Interleukin-1 (IL-1) was the first cytokine to be found useful as an adjuvant in amplifying the secondary antibody response to bovine serum albumin by a cell-mediated immunity via increasing production of interleukin-2 (IL-2). Previous studies have shown that recombinant bovine IL-1β is useful as an immunomodulator of bovine immune responses to viral infections (see Reddy et al, “Adjuvanicity of recombinant bovine interleukin-1β: influence on immunity, infection and latency in bovine herpes virus-1 infection,” Lymphokine Res. 9:295-300 (1990)). In these studies, r-BoIL-1β-treatment of calves increased antibody production against bovine herpes virus-1 (BHV-1), bovine virus diarrhea (BVD) and parainfluenza-3 (PI-3) viruses, enhanced cytotoxic responses to virally infected MDBK cells, decreased viral shedding of BHV-1 after challenge and had lower recrudescence of BHV-1 following dexamethasone injections. The reports suggested that recombinant bovine interleukin-1β can potentiate the activity of antigens when administered subcutaneously in an aqueous solution.

Clinical trials have been performed to assess the ability of cytokines such as interferon α (IFN-α) and interferon γ (IFN-γ) to improve the immunogenicity of hepatitis B vaccines in non-responsive subjects.

Subsequent research in immunology has examined the importance and activity of other cytokines such as, for example, interleukin-12 (see, for example, Y.-W. Tang et al., “Interleukin-12 Treatment during Immunization Elicits a T Helper Cell Type 1-like Immune Response in Mice Challenged with Respiratory Syncytial Virus and Improves Vaccine Immunogenicity,” J. Infectious Diseases 112:734-738 (1995); S. Morris et al, “Effects of IL-12 on in Vivo Cytokine Gene Expression and Ig Isotype Selection,” J. Immunology, pp. 1047-1056 (1994); J. Orange et al., “Effects of IL-12 on the Response and Susceptibility to Experimental Viral Infections,” J. Immunology, pp. 1253-1264 (1994); G. Trinchieri, “Interleukin-12 and its role in the generation of T_{H}1 cells,” Immunology Today 14(7):335-338 (1993); R. Gazzinelli et al., “Interleukin-12 is required for the T-lymphocyte-independent induction of interferon γ by an intracellular parasite and induces resistance in T-cell-deficient hosts,” Proc. Natl. Acad. Sci. USA 90:6115-6119 (July 1993); R. Locksley, “Commentary: Interleukin-12 in host defense against microbial pathogens,” Proc. Natl. Acad. Sci. USA 90:5879-5880 (July 1993); B. Graham et al., “Priming Immunization Determines T Helper Cytokine mRNA Expression Patterns in Lungs of Mice Challenged with Respiratory Syncytial Virus,” J. Immunology 151:2032-2040 (Aug. 15, 1993); J. Sypek et al., “Resolution of Cutaneous Leishmaniasis: Interleukin 12 Initiates a Protective T Helper Type 1 Immune Response,” J. Exp. Med. 177:1797-1802 (June 1993); F. Heinzel et al., “Recombinant Interleukin 12 Cures Mice Infected with Leishmania major,” J. Exp. Med. 177:1505-1509 (May 1993); C. Tripp et al., “Interleukin 12 and tumor necrosis factor α are costimulators of interferon γ production by natural killer cells in severe combined immunodeficiency mice with listeriosis, and interleukin 10 is a physiologic antagonist,” Proc. Natl. Acad. Sci. USA 90:3725-3729 (April 1993); R. Manetti et al., “Natural Killer Cell Stimulatory Factor (Interleukin 12 [IL-12]) Induces T Helper Type 1 (Th1)-specific Immune Responses and Inhibits the Development of Il-4-producing Th Cells,” J. Exp. Med. 177:1199-1204 (April 1993); C.-S. Hsieh et aL, “Development of T_{H}1 CD4⁺ T Cells Through IL-12 Produced by Listeria-Induced Macrophages,” Science 260:547-548 (April 23, 1993); P. Scott, “IL-12: Initiation Cytokine for Cell-Mediated Immunity,” Science 260:496-497 (Apr. 23, 1993); M. Gately et al., “Regulation of Human Cytolytic Lymphocyte Responses by Interleukin 12,” Cellular Immunology 143:127-142 (1992); A. D'Andrea et al., “Production of Natural Killer Cell Stimulatory Factor (Interleukin 12) by Peripheral Blood Mononuclear Cells,” J. Exp. Med. 176:1387-1398 (November 1992); B. Naume et al., “A comparative study of IL-12 (Cytotoxic Lymphocyte Maturation Factor)-, IL-2-, and IL-7-induced effects on Immunomagnetically purified CD56 NK cells,” J. Immunology 148:2429-2436 (Apr. 15, 1992); S. Chan et al., “Induction of Interferon γ Production by Natural Killer Cell Stimulatory Factor: Characterization of the Responder Cells and Synergy with Other Inducers,” J. Exp. Med. 173:869-879 (April 1991); and M. Kobayashi et al., “Identification and Purification of Natural Killer Cell Stimulatory Factor (NKSF), a Cytokine with Multiple Biologic Effects on Human Lymphocytes,” J. Exp. Med. 170:827-845 (September 1989)).

Interleukin-12 (hereinafter referred to as “IL-12”) has demonstrated adjuvant activity in eliciting a cell-mediated immunity against leishmaniasis in BALB/c mice (L. Afonso et al., “The Adjuvant Effect of Interleukin-12 in a Vaccine Against Leishmania major,” Science 263:235-237 (Jan. 14, 1994)). The conferral of protection against L. major was based on the activity of IL-12 to induce the development of leishmanial-specific CD4⁺ T_{H}1 (T helper) cells. U.S. Pat. No. 5,571,515 (Scott et al.) and related divisions, U.S. Pat. Nos. 5,723,127 and 5,976,539, describe the use of IL-12 as an adjuvant against leishmaniasis by enhancing the cell-mediated immune response to an antigen comprising the protozoan parasite. Based on the description of the use of IL-12 as an adjuvant in the leishmaniasis model and with a cancer vaccine, U.S. Pat. No. 5,723,127 is directed to antigenic compositions of selected antigens and IL-12, and the method for increasing the ability of the compositions to elicit the host's cell-mediated immune response to the selected antigens. U.S. Pat. No. 5,976,539 is drawn to a composition of an antigen selected from cancer cells or cancer cells transfected with a selected antigen and IL-12 and the method of use thereof. A further related continuation in this series, U.S. Pat. No. 6,168,923 B1 (Scott et al.), claims a composition comprising an antigen consisting of a pathogenic microorganism and IL-12 which elicits a vaccinated host's cell-mediated immune response against the microorganism and a method of administering IL-12 to increase the ability of an immunogenic composition to elicit a vaccinated host's cell-mediated immune response.

U.S. Pat. No. 5,665,347 (Metzger et aL) discloses that, in addition to activation of T_{H}1 (T helper) cells, IL-12 inhibits the functional activity of B 1 cell activity but not B2 cells, and B1 cells possess an IL-12 receptor. Patentees suggest that IL-12 may find use in treatment of B1 cell disorders like chronic lymphocytic leukemia, lymphomas and infectious mononucleosis.

U.S. Pat. No. 5,817,637 (Weiner et al.) relate to a pharmaceutical immunizing kit that uses genetic material as the immunizing agent in two separate inoculants. A third inoculant contains bupivacaine that may be combined with other response enhancing agents like transfecting, replicating or inflammatory agents, for example, lectins, growth factors, cytokines (such as α-interferon, γ-interferon, IL-1, IL-2, IL-4, IL-6, IL-8, IL-10, IL-12, etc.) and lymphokines.

U.S. Pat. No. 5,985,264 (Metzger et aL) concern the method of enhancing an immune response to a pathogen in a neonatal host comprising the administration of IL-12 and an antigen to induce memory for protective responses as an adult. The neonatal host is mammalian, for example, human, murine, feline, canine, bovine or porcine, and includes the fetus as well as newborn to about 2 years after birth. The antigen is described as bacteria (e.g., S. pneumoniae, N. meningiditis, H. influenza), viruses (e.g., hepatitis, measles, poliovius, human immunodeficiency, influenza, parainfluenza, respiratory syncytial), parasites (e.g., Leishmania, Schistosomes) and fungi (e.g., Candida, Aspergillus).

U.S. Pat. No. 5,744,132 (Warne et al.) describes compositions and methods for providing concentrated preparations of IL-12 in a frozen, liquid or lyophilized formulation of the IL-12 protein, polysorbate, a cryoprotectant, bulking agents and buffering agents. U.S. Pat. No. 5,853,714 (Deetz et al.) provides a method for purification of IL-12 using a hydrophobic interaction chromatography resin to make IL-12 free of contaminants such as host cell proteins and viruses.

In addition to the above art, there are several patents and publications in this crowded field that describe the use of IL-12 with certain antigens, for example, as an adjuvant in paramyxoviridae vaccines (U.S. Pat. No. 6,071,893, Graham et al.), for enhancing oral tolerance and treating autoimmune disease (WO 98/16248), for treating inflammation (U.S. Pat. No. 5,674,483, Tu et al.), as an adjuvant in Bordetella pertussis vaccines (WO 97/45139) or as a co-adjuvant with IL-13 in vaccines containing antigens such as influenza A, HIV, tetanus toxoid, etc. (WO 98/31384) and the like. Further research has provided a variety of animal cytokines and the methods to produce them, for example, feline IL-12 (C. Leutenegger et al., “Immunization of Cats against Feline Immunodeficiency Virus (FIV) Infection by Using Minimalistic Immunogenic Defined Gene Expression Vector Vaccines Expressing FIV gp140 Alone or with Feline Interleukin-12 (IL-12), IL-16, or a CpG Motif,” J. Virology 74(22):10447-10457 (November 2000) and WO 01/04155 A2), avian IL-15 (WO 97/14433), ovine IL-5 or IL-12 (WO 97/00321), to name just a few.

Other research, including some of the publications described hereinabove, has focused on particular vaccine formulations and the methods of making them. U.S. Pat. No. 5,242,686 (Chu et al.), for instance, is directed to a process for preparing a feline vaccine composition useful against chlamydia infections. The inactivated mammalian chlamydial cells or antigens may be combined with an immunogenically suitable adjuvant and a physiologically acceptable carrier. The patent lists the adjuvant, for example, as surfactants, polyanions, polycations, peptides, tuftsin, oil emulsions, immunomodulators such as interleukin-1, interleukin-2 and interferons, acrylic acid copolymers such as ethylene/maleic anhydride copolymer, copolymers of styrene with a mixture of acrylic acid and methacrylic acid or a combination thereof.

U.S. Pat. No. 5,733,555 (Chu) and its continuation, U.S. Pat. No. 5,958,423 concern a vaccine composition for immunizing an animal against infection caused by Bovine Respiratory Syncytial Virus (“BRSV”) which contains a modified live BRSV alone or in combination with a Bovine Rhinotracheitis Virus IV, a Bovine Viral Diarrhea Virus and a Parainfluenza 3 Virus, an adjuvant and a pharmaceutically acceptable carrier. The composition elicits protective immunity after a single administration via cell-mediated immunity, secretory immunoglobulin A immunity and a combination thereof. The adjuvant may further comprise a surfactant such as polyoxyethylene sorbitan monooleate. The patents list other adjuvants such as squalane, squalene, block copolymers, saponin, detergents, Quil A, mineral oils, vegetable oils, interleukins such as interleukin-1, interleukin-2 and interleukin-12, tumor necrosis factor, interferons, combinations such as saponin and aluminum hydroxide or Quil A and aluminum hydroxide, liposomes, iscom adjuvant, synthetic glycopeptides such as muramyl dipeptides, dextran, carboxypolymethylene, EMA®, acrylic copolymer emulsions such as Neocryl® A640 or mixtures thereof.

However, it has not been described or exemplified in the art that IL-12 or other immunomodulators can effectively and markedly enhance the immunogenicity of weak, immunosuppressive or potentially toxic antigens when specifically co-administered with immunoadjuvants.

It is therefore an important object of the present invention to provide a highly unique vaccine possessing significantly improved immunogenicity in mammals and birds that is comprised of weak or immunosuppressive antigens, or antigens with a narrow margin of safety, in conjunction with the novel combination comprising the immunomodulators and the immunoadjuvants of this invention.

Another object is to provide a new method of using the combination comprising the immunomodulators and the immunoadjuvants or the vaccine that contains the combination to substantially improve the immunogenicity of the vaccine by inducing a stronger stimulation on cell-mediated immunity including T memory cells and to provide a longer duration of immunity thereby requiring smaller or less frequent dosages of antigens over time and lessening side effects or potential for toxicity.

A further object is to provide a new method of potentiating, accelerating or extending the immunological activity of an antigen in an avian or mammalian species.

Further purposes and objects of the present invention will appear as the specification proceeds.

The foregoing objects are accomplished by providing a combination of immunomodulators and immunoadjuvants, and a vaccine in which an immunomodulator is co-formulated with an immunoadjuvant and a viral, bacterial, parasitic or fungal antigen. The product of this invention produces a highly improved immunological response to the antigen as compared to classical vaccines and other combinations comprising a cytokine by itself. The background of the invention and its departure from the art will be further described hereinbelow.

### BRIEF SUMMARY OF THE INVENTION

The present invention involves an improved vaccine formulation that comprises an effective immunizing amount of an antigen, an immunomodulator and one or more immunoadjuvants in which the immunogenicity or physiological efficacy of the vaccine is significantly enhanced. The invention includes the novel combination composition comprising the immunomodulators and the immunoadjuvants that markedly improves the immunological response of a vaccinated host to the antigen. Also, the present invention concerns a novel method for potentiating, accelerating or extending the immunogenicity of weak, immunosuppressive or marginally safe antigens which comprises administering to an avian or mammalian species a pharmacologically effective amount of the aforesaid combination composition or an effective vaccinating amount of the aforedescribed vaccine composition.

### BRIEF DESCRIPTION OF THE DRAWINGS

Not Applicable

### DETAILED DESCRIPTION OF THE INVENTION

In accordance with the present invention, the novel vaccine composition comprises an effective immunizing amount of an antigen, an immunomodulator, one or more immunoadjuvants and a pharmaceutically acceptable carrier. Surprisingly, the incorporation of the immunomodulator and the immunoadjuvant(s) into vaccines significantly potentiates the immunogenicity and physiological efficacy of the antigenic substance. The unique combination of the immunomodulator and immunoadjuvants has beneficial application for increasing the biological activity of numerous antigens.

The antigen encompasses a wide variety of infectious agents contemplated by those of ordinary skill in the pharmaceutical or veterinary arts. The infectious agent, for example, may be viral, bacterial or fungal in nature. Other infectious agents include, but are not limited to, parasites, tumor antigens and antigens of other pathological diseases. The particular antigen or combination of antigens to be employed in the vaccine composition will depend upon the species to be vaccinated and the desired results.

The antigen is incorporated with the immunomodulator and the immunoadjuvant in varying amounts and usually ranges from about 0.0001% to about 1.0% by weight. Examples of typical viral antigens include, but are not limited to, Bovine Respiratory Syncytial Virus, herpes simplex virus type 1 (HSV), bovine virus diarrhea (BVD), parainfluenza-3 virus (PI), canine parvovirus (CPV), canine parainfluenza virus (CPI), canine adenovirus type II (CAV-2), canine adenovirus (CDV), canine coronavirus (CCV), rabies virus (particularly for, but not limited to, canine rabies vaccines), feline immunodeficiency virus (FIV), feline leukemia virus (FeLV), feline coronavirus (etiologic agent of feline infectious peritonitis (FIP)), Porcine Reproductive and Respiratory Syndrome (PRRS) Virus, chicken herpes virus (etiologic agent of Marek's Disease), etc. Typical bacterial antigens include, but are not limited to, Chlamydia, Ehrlichia, Pasteurella, Haemophilus, Salmonella, Staphylococcus, Streptococcus, Borrelia, Mycoplasma (for example, swine disease of Mycoplasma hyopneumoniae), etc. Typical parasitic antigens include, but are not limited to, Leptospira, Coccidia, Hemosporidia, Amoebida, Trypanosoma, Leishmania, Giardia, Histomonas, etc. Typical fungal antigens include, but are not limited to, Coccidioides, Histoplasma, Blastomyces, Aspergillus, Cryptococcus, etc.

The immunomodulator is present in the improved vaccine of the invention in varying amounts and usually ranges from about 0.00001% to about 0.01% by weight. Examples of suitable immunomodulators include, but are not limited to, cytokines such as IL-2, IL-4, IL-6, IL-8, IL-10, IL-12, etc., interferons such as α-interferon or γ-interferon, tumor necrosis factor, transforming growth factor, colony stimulating factor and the like, or a combination thereof. Desirably, the immunomodulator comprises a cytokine. In a preferred embodiment, the immunomodulator is interleukin-12 and most preferably, the homologous animal interleukin-12 such as, for example, canine IL-12 is employed in canine vaccines; feline IL-12 is employed in cat vaccines; equine IL-12 is employed in horse vaccines and so forth. Human IL-12 or murine IL-12, such as recombinant human IL-12 (commercially available from Genetics Institute, Inc., Cambridge, Mass.) or recombinant murine IL-12 (commercially available from various suppliers, for example, Research Diagnostics, Inc., Flanders, N.J. and Cambridge Bioscience, Cambridge, England; see also D. Schoenhaut et al., “Cloning and Expression of Murine IL-12,” J. Immunology 248(1):3433-3440 (Jun. 1, 1992)), may suitably be employed for a variety of animal species although the immunopotentiation effect may not be as great as the homologous animal IL-12 in some animal vaccines.

One or more immunoadjuvants are present in the improved vaccine of the invention in varying amounts and usually range from about 0.05% to about 50% by weight. Examples of suitable immunoadjuvants include, but are not limited to, metabolizable oils of plant or animal origin such as squalene (2,6,10,15,19,23-hexamethyl-2,6,10,14,18,22-tetracosahexaene) or preferably, squalane (2,6,10,15,19,23-hexamethyl-tetracosane); block copolymers, for example, polyoxypropylene-polyoxyethylene block copolymers such as Pluronic® (commercially available from BASF Corporation, Mount Olive, N.J.); saponin such as Quil A (commercial name of a purified form of Quillaja saponaria, available from Iscotec AB, Sweden and Superfos Biosector a/s, Vedbaek, Denmark); ethylene/maleic copolymers such as EMA-31® (a linear ethylene/maleic anhydride copolymer having approximately equal amounts of ethylene and maleic anhydride, having an estimated average molecular weight of about 75,000 to 100,000, commercially available from Monsanto Co., St. Louis, Mo.); acrylic acid copolymers; acrylic acid copolymer emulsions such as Neocryl® (an uncoalesced aqueous acrylic acid copolymer of acrylic acid and methacrylic acid mixed with styrene, commercially available from Polyvinyl Chemicals, Inc., Wilmington, Mass.); mineral oil emulsions such as MVP® (an oil-in-water emulsion of light mineral oil, commercially available from Modern Veterinary Products, Omaha, Nebr.) or mixtures thereof.

The preferred polyoxypropylene-polyoxyethylene block copolymers of the present invention include varying amounts of polyoxypropylene and polyoxyethylene. Desirably, the block copolymer comprises polyoxyethylene in the amount of about 10-20% of the total molecule and polyoxypropylene in an average molecular weight of about 3250 to 4000.

The ethylene/maleic copolymers of the invention are typically water soluble, white, free-flowing powders having the following properties: a true density of about 1.54 g/mL, a softening point of about 170° C., a melting point of about 235° C., a decomposition temperature of about 274° C., a bulk density of about 20 lbs/ft³ and a pH of about 2.3 (1% solution).

A preferred acrylic acid copolymer emulsion of the invention is Neocryl® A640 which comprises an aqueous acrylic acid copolymer having a pH of 7.5, viscosity of 100 eps (Brookfield, 25° C.), a weight per gallon of 8.6 pounds as supplied containing 40% solids by weight, 38% solids by volume and an acid number of 48. Specifically, Neocryl® A640 is a latex emulsion of an uncoalesced aqueous acrylic acid copolymer of acrylic acid and methacrylic acid mixed with styrene. Other useful products include, but are not limited to, Neocryl® A520 and A625, and the like.

Preferred combinations of immunomodulators and immunoadjuvants comprise a mixture of the homologous animal IL-12, squalane and a polyoxypropylene-polyoxyethylene block copolymer; a mixture of the homologous animal IL-12 and saponin; and a mixture of the homologous animal IL-12, EMA-31® and Neocryl® A640 with or without a mineral oil emulsion. Recombinant human or murine IL-12 may be substituted for the homologous animal L-12, though a partial immunopotentiation effect may be elicited. Under those certain circumstances, the efficacy or potency can be readily determined through routine tests and then the dosage of the active ingredient can be appropriately titrated in the patient or animal as needed.

A pharmaceutically acceptable carrier is also present in the vaccine composition of this invention in varying amounts. The amount of the nontoxic, inert carrier, of course, will be dependent upon the amounts selected for the other ingredients, the desired concentration of the active antigenic substance, the selection of the vial, syringe or other conventional vehicle size, etc. The carrier can be added to the vaccine at any convenient time. In the case of a lyophilized vaccine, the carrier can, for example, be added immediately prior to administration. Alternatively, the final product can be manufactured with the carrier. Examples of appropriate carriers include, but are not limited to, sterile water, saline, buffers, phosphate-buffered saline, buffered sodium chloride, vegetable oils, Minimum Essential Medium (MEM), MEM with HEPES buffer, etc.

Optionally, the composition may contain conventional, secondary adjuvants in varying amounts depending on the adjuvant and the desired result. The customary amount ranges from about 0.02% to about 20% by weight or provides from about 1 μg to about 50 μg per dose, depending upon the other ingredients and desired effect. Examples of suitable secondary adjuvants include, but are not limited to, stabilizers; emulsifiers; aluminum hydroxide; aluminum phosphate; pH adjusters such as sodium hydroxide, hydrochloric acid, etc.; surfactants such as Tween® 80 (polysorbate 80, commercially available from Sigma Chemical Co., St. Louis, Mo.); liposomes; iscom adjuvant; synthetic glycopeptides such as muramyl dipeptides; extenders such as dextran or dextran combinations, for example, with aluminum phosphate; carboxypolymethylene; bacterial cell walls such as mycobacterial cell wall extract; their derivatives such as Corynebacterium parvum; Propionibacterium acne; Mycobacterium bovis, for example, Bovine Calmede Guern (BCG); vaccinia or animal poxvirus proteins; subviral particle adjuvants such as orbivirus; cholera toxin; N,N-dioctadecyl-N′,N′-bis(2-hydroxyethyl)-propanediamine (avridine); monophosphoryl lipid A; dimethyldioctadecylammonium bromide (DDA, commercially available from Kodak, Rochester, N.Y.); synthetics and mixtures thereof Desirably, aluminum hydroxide is admixed with other secondary adjuvants or an immunoadjuvant such as Quil A. Examples of suitable stabilizers include, but are not limited to, sucrose, gelatin, peptone, digested protein extracts such as NZ-Amine or NZ-Amine AS. Examples of emulsifiers include, but are not limited to, mineral oil, vegetable oil, peanut oil and other standard, metabolizable, nontoxic oils useful for injectables or intranasal vaccines.

For purposes of this invention, these adjuvants are identified herein as “secondary” merely to contrast with the above-described immunoadjuvant that is an essential ingredient in the vaccine for its effect in combination with the immunomodulator to significantly increase the humoral immune response of the mammal or the bird to the antigenic substance. The secondary adjuvants are primarily included in the vaccine formulation as processing aids although certain adjuvants do possess immunologically enhancing properties to some extent and have a dual purpose.

As needed, conventional preservatives can be added to the vaccine in effective amounts ranging from about 0.0001% to about 0.1% by weight. Depending on the preservative employed in the formulation, amounts below or above this range may be useful. Typical preservatives include, for example, potassium sorbate, sodium metabisulfite, phenol, methyl paraben, propyl paraben, thimerosal, etc.

The choice of inactivated, modified or other type of vaccine and method of preparation of the improved vaccine formulation of the present invention are known or readily determined by those of ordinary skill in the art. As an illustration of the preparation of inactivated vaccines, for example, the immunomodulator, preferably the homologous animal IL-12, is mixed with one or more antigens, one or more immunoadjuvants and, optionally, one or more secondary adjuvants. The antigens may be the inactivated FIV, FeLV, E. canis, CCV, Leptospira species, etc. As a further illustration, the immunomodulator, preferably the homologous animal IL-12, is mixed with antigens in the presence or absence of the immunoadjuvants or secondary adjuvants to prepare modified vaccines. The antigens in this case may be BRSV, S. equi, CPV, CAV-2, CDV, CPI, etc. It is appreciated, however, that the vaccines of the present invention may be made by a variety of standard techniques well known to those in the formulations art and are not limited by the illustrations described herein.

The combination comprising the immunomodulators and the immunoadjuvants may be prepared and administered as a separate product. A pharmacologically effective amount of this immunogenicity enhancing composition may be given, for example, parenterally, orally or otherwise, to a mammal or a bird before, concurrently with, sequentially to or shortly after the administration of a weak, immunosuppressive or marginally safe antigen in order to potentiate, accelerate or extend the immunogenicity of the antigen. Typically, the immunogenicity enhancing composition will be administered within 24 hours before the start of the vaccination process and, preferably within four hours before or concurrently with the vaccination. If vaccination requires more than one dose of the antigenic substance, then the immunogenicity enhancing composition may be administered in sequential fashion with the administration of the vaccine. Although less effective, the immunogenicity enhancing composition may be given after the vaccine to boost the immunity against the antigen, but rarely beyond 24 hours.

When given separately from the vaccine, the combination may further comprise a pharmaceutically acceptable carrier and optionally, secondary adjuvants which are described herein. Both the immunomodulator and the immunoadjuvant may be present in varying amounts, typically in a unit dosage container. While the dosage of the combination depends upon the antigen, species, body weight of the host vaccinated or to be vaccinated, etc., the dosage of a pharmacologically effective amount of the immunomodulator will usually range from about 0.1 μg to about 100 μg per dose and, preferably, about 5 μg to about 50 μg per dose. The immunoadjuvant will typically range from about 1 μg to about 25 μg per dose. Although the presence and the amount of the particular immunoadjuvant in the combination will influence the amount of the immunomodulator necessary to improve the immune response, it is contemplated that the practitioner can easily adjust the effective dosage amount of the immunomodulator through routine testing to meet the particular circumstances.

When the homologous animal IL-12 is employed, the amount of the immunomodulator in the vaccine may be significantly reduced due to its potency. For small animals like dogs, cats, etc., a range of about 0.02 μg to about 2 μg per dose of homologous animal IL-12 is typically used, about 0.1 μg to about 1 μg per dose of the animal IL-12 is preferably used and about 0.5 μg per dose is more preferably used in the combination composition of the invention. For large animals like horses, cattle, swine, etc., a range of about 0.1 μg to about 5 μg per dose of animal IL-12 is typically used and about 0.5 μg to about 2.5 μg per dose is preferably used. It is appreciated that amounts below and above these given ranges may find their respective uses in the smaller birds and extremely large animals. To retain biological activity, it is also recommended that the animal IL-12 be added to the vaccine or unit dosage form immediately prior to use.

As a non-limiting example, a suitable canine vaccine may comprise the Ebony strain of E. canis at a concentration/dose of 1×10⁵ TCID₅₀; B. burgdorferi IPS at a concentration/dose of 5×E7; B. burgdorferi B-31 at a concentration/dose of 5×E8; 5% v/v of emulsigen SA; 1% v/v of EMA-31®; 3% v/v of Neocryl® A640; 1:20,000 concentration of thimerosal (5%); a suitable amount of 1× MEM diluent and canine IL-12 at a concentration per dose of approximately 0.5 μg or human IL-12 at a concentration of approximately 10 μg per dose.

The present invention further embraces the novel method for potentiating, accelerating or extending the immunogenicity of weak, immunosuppressive or marginally safe antigens which comprises administering to an avian or mammalian species a pharmacologically effective amount of the immunogenicity enhancing composition or an effective vaccinating or immunizing amount of the vaccine formulation described herein. Potentiating the immunogenicity of the weak, immunosuppressive or marginally safe antigens involves improving the potency of the antigens. Accelerating the immunogenicity refers to speeding up the onset of action. Extending the immunogenicity means lengthening the duration of activity.

As a general rule, the vaccine of the present invention is conveniently administered parenterally (subcutaneously, intramuscularly, intravenously, intradermally or intraperitoneally), intrabuccally, intranasally, transdermally or orally. The route of administration contemplated by the present invention will depend upon the antigenic substance and the co-formulants. For instance, if the vaccine contains saponins, while non-toxic orally or intranasally, care must be taken not to inject the sapogenin glycosides into the blood stream as they function as strong hemolytics. Also, many antigens will not be effective if taken orally. Preferably, the vaccine is administered subcutaneously, intramuscularly or, in the case of S. equi and others, intranasally.

The dosage of the vaccine will be dependent upon the selected antigen, the route of administration, species, body weight and other standard factors. It is contemplated that a person of ordinary skill in the art can easily and readily titrate the appropriate dosage for an immunogenic response for each antigen to achieve the effective immunizing amount and method of administration.

Advantageously, by using the antigen and an immunomodulator such as a cytokine, preferably the homologous animal IL-12, in combination with immunoadjuvants in a vaccine formulation, the improved vaccine is highly antigenic, eliciting a stronger stimulation of T memory cells than had been achievable in the past. The serum antibody titers to antigenic substances after vaccination with the formulation of the present invention are much higher than the titers induced by the same formulation in the absence of the immunomodulator. For instance, a previous study showed that the serum antibody titers to BRSV at 14 days after vaccination with BRSV adjuvanted with a mixture of squalane and a polyoxypropylene-polyoxyethylene block copolymer were about 1:125. Surprisingly, the serum antibody titers to BRSV at 14 days after vaccination with BRSV mixed with squalane, a polyoxypropylene-polyoxyethylene block copolymer and recombinant human IL-12 are distinctly higher at about 1:395, and remarkably still higher at about 1:366 after 28 days. The significantly enhanced immunogenicity, the accelerated onset of action and the extended duration of immunity are evidenced by heightened serum antibody titers (i.e., humoral immune response) and stronger stimulation of T memory cells. The substantial improvement in the efficacy of the vaccine of this selective invention gives a profound departure from the state of the art.

As used herein, the “CFU” stands for colony forming units. An “infectious unit” of BRSV, for example, is defined as the TCID₅₀. “TCID₅₀” or 50% Tissue Culture Infectious Dose is defined as the dose which infects 50% of the tissue culture. For example, when a solution containing an antigen is diluted 1:100, 1 infectious unit equals the amount which affects 50% of the tissue culture. In the case of BRSV, the TCID₅₀ is the amount of virus which is required to infect or kill 50% of the tissue culture cells. The term “cell-mediated immunity” includes the stimulation of T-Helper Cells, T-Killer Cells and T-Delayed Hypersensitivity Cells as well as the stimulation of macrophage, monocyte and other lymphokine and interferon production. The presence of cell-mediated immunity can be determined by conventional in vivo and in vitro assays. Local immunity such as secretory IgA can be determined by conventional ELISA or IFA assays showing a serum neutralizing antibody titer of 1 to 2 or greater. The cell-mediated or local immunity elicited according to the present invention is specific to or associated directly with the antigen. The term “mammal” refers to humans, cattle, cows, sheep, deer, horses, swine, goats, dogs, cats and the like. The term “avian” refers to poultry such as chickens or turkey and other types of domesticated or wild birds. Although veterinary use in animals is preferred, it is contemplated that the immunogenicity enhancing and vaccine compositions described herein may find beneficial medical use.

A further understanding of the present invention can be obtained from the following non-limiting examples. However, the examples are set forth only for the illustration of certain aspects of the invention and are not to be construed as limitations thereon. It is to be understood that the examples do not purport to be wholly definitive as to conditions and scope of this invention. It should be further appreciated that when typical reaction conditions (e.g., temperature, reaction times, etc.) have been given, the conditions both above and below the specified ranges can also be used, though generally less conveniently. The following experimental studies employ recombinant human IL-12 that is obtained from Genetics Institute, Inc., Cambridge, Mass., a wholly-owned subsidiary of Wyeth, Madison, N.J. Unless otherwise expressed, the examples are conducted at room temperature (about 23° C. to about 28° C.) and at atmospheric pressure, all parts and percents referred to herein are by weight, and all temperatures are expressed in degrees centigrade.

### EXAMPLE 1

### Effect of Adjuvant on Immunogenicity of Horse Vaccine

A study is performed to determine the effect of certain adjuvants on the immunogenicity of an inactivated vaccine of Streptococcus equi. To prepare the adjuvants, stock solutions of recombinant human IL-12 (4.45 mg/mL), saponin, a stabilizer for modified live vaccines (SGGK-3, 25% v/v) and a bacterial growth medium (Modified Todd Hewitt Broth, MTHB) are used. Three adjuvant blends are made to approximate 10 μg of IL-12 per dose, 50 μg of IL-12 per dose and 10 μg of IL-12 plus 5 mg of saponin per dose.

To prepare the adjuvant blend comprising about 10 μg of IL-12 per dose, a rehydration diluent is made by adding about 0.056 mL of IL-12 to about 49.719 mL of a sufficient quantity of water to total 50 mL. An adjustment diluent is then made by adding about 0.056 mL of IL-12 to about 12.5 mL of SGGK-3 (25% v/v) mixed with about 37.444 mL of MTHB.

To prepare the adjuvant blend comprising about 50 μg of IL-12 per dose, a rehydration diluent is made by adding about 0.281 mL of IL-12 to about 49.719 mL of a sufficient quantity of water to total 50 mL. An adjustment diluent is then made by adding about 0.281 mL of IL-12 to about 12.5 mL of SGGK-3 (25% v/v) mixed with about 37.219 mL of MTHB.

To prepare the adjuvant blend comprising about 10 μg of IL-12 plus 5 mg of saponin per dose, a rehydration diluent is made by adding about 0.056 mL of IL-12 to about 0.625 mL of saponin and the mixture to about 49.319 mL of a sufficient quantity of water to total 50 mL. An adjustment diluent is then made by adding about 0.056 mL of IL-12 to about 0.625 mL of saponin and the mixture to about 12.5 mL of SGGK-3 (25% v/v) mixed with about 37.819 mL of MTHB.

For the preparation of each vaccine, one vial of Pinnacle® I.N. (an intranasal equine Strangles vaccine, commercially available from Fort Dodge Animal Health, Inc., a veterinary division of Wyeth, Madison, N.J.) is rehydrated with about 2.5 mL of rehydrating diluent. Ten doses of vaccine are prepared for each group (approximately 20 mL of vaccine). After rehydrating the vaccine, about 0.467 mL of rehydrated vaccine is added to about 19.533 mL of adjustment diluent to obtain an amount of approximately 1×10⁷ CFU per dose.

All horses subjected to the test vaccines are vaccinated two times with three weeks between vaccinations. The vaccine is administered intranasally with a syringe connected to a catheter of about 5.5 inches in length. The first vaccination is administered into the left nostril and the second vaccination is administered into the right nostril. All of the horses in the control group are vaccinated with a commercially available Streptococcus equi vaccine (Stepguard® with Havlogen®, an adjuvant consisting of carboxypoly-methylene, manufactured by Bayer Animal Health, Inc., an agricultural division of Bayer Corporation) and receive two vaccinations three weeks apart. The commercial vaccine is administered intramuscularly according to the manufacturer's instruction.

Five horses are not vaccinated and, instead, are inoculated with 1 mL (approximately 5×10⁸ CFU/mL) of the S. equi CF-32 strain into each nostril 5 days before the contact challenge. A syringe with a catheter of about 5.5 inches in length is used to inoculate the horses. The five horses are observed daily from two days before to five days post challenge for clinical signs and rectal temperature. Nasal swabs are collected daily after challenge to monitor S. equi shedding. Twenty-one days post second vaccination, all the vaccinated horses are commingled with the five direct challenged horses. The animals are observed daily from −2 days to 0 days post challenge (DPC) to establish a baseline and 1 to 28 days DPC for various clinical signs. Animals are observed additionally on 30, 33 and 36 DPC.

The below Table 1 shows that adjuvants IL-12 (approximately 50 μg IL-12/dose) and a combination of IL-12 with saponin are relatively better immunostimulators compared to the rest of the adjuvants used in the study as demonstrated by average clinical score, incidence of local lymph node abscess, S. equi shedding and disseminated abscess. Horses in these two groups show about 35% to about 40% reduction in the incidence of disseminated abscess and about 23% to about 40% reduction of the average clinical score as compared to the group receiving the commercial vaccine without IL-12 or the combination of IL-12 and saponin.

TABLE 1
Results of S. Equi Study

| | Total | No. of | No. of Horses | % Reduction of | | % Reduction of |
|---|---|---|---|---|---|---|
| | Horses | Horses with | with | Horses with | Average | Average Clinical |
| | per | Local | Disseminated | Disseminated | Clinical | Score Compared |
| Adjuvant | Group | Abscess | Abscesses | Abscesses¹ | Score¹ | to Bayer Group |
| SP Oil | 5 | 5 | 2 | 20% | 65.6 | 13% |
| IL-12 | 5 | 5 | 1 | 40% | 59.6 | 14% |
| (10 μg) | | | | | | |
| IL-12 | 5 | 4 | 1 | 40% | 52.8 | 23% |
| (50 μg) | | | | | | |
| IL-12 | 4 | 3 | 1 | 35% | 47.5 | 40% |
| (10 μg) + Saponin | | | | | | |
| Carbopol | 5 | 5 | 2 | 20% | 61.8 | 21% |
| DDA + | 4 | 4 | 2 | 10% | 65.2 | 17% |
| DEAE Dex³ | | | | | | |
| Bayer Vaccine | 5 | 5 | 3 | N/A² | 78.6 | N/A |

¹Percentage of reduction of disseminated abscesses and average clinical score is measured for each group compared to Bayer group.
²“N/A” means not applicable.
³“DDA” is dimethyldioctadecylammonium bromide and “DEAE Dex” is diethylaminoethyl-dextran.

### EXAMPLE 2

### Effect of Adjuvant on Immunogenicity of Cattle Vaccine

A study is performed to determine the effect of a certain adjuvant on the immunogenicity of a modified live vaccine of BRSV (Bovine Respiratory Syncytial Virus). To prepare the adjuvant, stock solutions of SP oil adjuvant (5% v/v) and recombinant human IL-12 (about 1,260 μg per mL) are used.

SP oil is prepared by mixing 20 mL of Pluronic® L121 (a polyoxypropylene-polyoxyethylene block copolymer, commercially available from BASF Corporation, Mount Olive, N.J.), 40 mL of squalane, 3.2 mL of polysorbate 80 and 936.8 mL of a buffered salt solution and homogenizing the mixture until a stable mass or emulsion is formed. Prior to homogenation, the ingredients or mixture is autoclaved. The emulsion is further sterilized by filtration. Formalin and thimerosal are added to a final concentration of 0.2% and dilution of 1:10,000, respectively.

The adjuvant blend, which comprises about 5% v/v of SP oil plus about 10 μg of IL-12 per dose, is made by adding about 0.278 mL of IL-12 to about 69.722 mL of 5% v/v SP oil to make about 70 mL of about 5% v/v SP oil plus about 10 μg/dose of IL-12 adjuvant.

For preparation of the vaccine, BRSV are grown in MDBK cells (Madin-Darby Bovine Kidney cells; the MDBK cell line is derived from a kidney of a normal adult steer) and are harvested 6 days after inoculation. The vaccine cake is blended at BRSV titer of about 10^{5.7} TCID₅₀ per dose with MEM and then is lyophilized. The lyophilized vaccine cake is then rehydrated with the above-described IL-12 containing adjuvant diluent to make the final vaccine preparation.

Nine calves, about 6 months of age, are vaccinated with the BRSV vaccine subcutaneously, leaving seven calves as the control group. Serum antibody response is measured by detecting the specific antibody to BRSV. The efficacy of the vaccines is demonstrated by challenging the vaccinates and the controls with virulent BRSV 28 days post vaccination.

The modified live BRSV vaccines adjuvanted with SP oil+IL-12 induced a very high titer antibody response (about 1:366 at 28th day post vaccination) to BRSV. After the virulent BRSV challenge, the severity of the disease is reduced in the vaccinated group compared with the control group (about 53% reduction). This indicates that the SP oil+IL-12 adjuvant used in this study is compatible with the BRSV modified virus vaccine and can significantly enhance the efficacy of the BRSV modified virus vaccine.

The below Table 2 shows the antibody response to BRSV of calves vaccinated with modified live BRSV and IL-12 containing adjuvant.

TABLE 2
Antibody Response to BRSV

| | Number of | | | 28 DPV/ | | |
|---|---|---|---|---|---|---|
| Group | Animals | 0 DPV | 14 DPV | 0 DPC | 7 DPC | 14 DPC |
| Vaccinates | 9 | <5 | 625 | 366 | 150 | 2,420 |
| Control | 7 | <5 | <5 | <5 | <5 | 125 |

The below Table 3 shows the disease reduction of calves vaccinated with modified live BRSV and IL-12 containing adjuvant after virulent BRSV challenge.

TABLE 3
Disease Reduction

| | Number of | Average | Disease |
|---|---|---|---|
| Group | Animals | Total Score | Reduction |
| Vaccinates | 9 | 2.7 | 53%¹ |
| Control | 7 | 5.7 | N/A² |

¹Disease reduction is the percentage of calves which do not show the disease after challenge as compared to controls.
²“N/A” means not applicable.

### EXAMPLE 3

### Effect of Adjuvant on Efficacy of Dog Vaccine

A study is performed to determine the effect of a certain adjuvant on the immunogenicity of a monovalent vaccine, killed bacterin, of Ehrlichia canis. To prepare the adjuvant, stock solutions of recombinant human IL-12 (4.45 mg/mL), EMA-31® (1% v/v, a linear ethylene/maleic anhydride copolymer having approximately equal amounts of ethylene and maleic anhydride, having an estimated average molecular weight of about 75,000 to 100,000, commercially available from Monsanto Co., St. Louis, Mo.) and Neocryl® A640 (3% v/v, a latex emulsion of an uncoalesced aqueous acrylic acid copolymer of acrylic acid and methacrylic acid mixed with styrene, having a pH of 7.5, viscosity of 100 eps (Brookfield, 25° C.), a weight per gallon of 8.6 pounds as supplied containing 40% solids by weight, 38% solids by volume and an acid number of 48, commercially available from Polyvinyl Chemicals, Inc., Wilmington, Mass.) are used. A working solution of IL-12 is prepared in a dilution buffer comprising phosphate buffered saline without magnesium and calcium. Forty-five μL of the IL-12 stock solution is added to 9.955 μL of the dilution buffer. The final concentration of the diluted IL-12 working solution is 20 μg/mL.

For preparation of the vaccine, approximately 1×10⁴ or 1×10⁵ TCID₅₀ of an inactivated Ebony strain of E. canis is blended with 1% v/v of EMA-31® and 3% v/v of Neocryl®. Two percent of thimerosal is added to the vaccine at a level of about 1:20,000 as preservative. The diluted IL-12 working solution in the amount of 500 μL per dose is mixed with the vaccine prior to injection. The vaccine for group 4 as shown in Table 4 below is blended with 100 μg/dose of Bovine Calmede Guern (BCG) bacterin.

Thirty-five dogs are randomized into six groups including four vaccination groups and two control groups. The vaccinates are vaccinated with a monovalent Ebony strain of E. canis vaccine at two antigen levels. As shown in Table 4 below, group 2 is vaccinated with the antigen level of approximately 1×10⁴ TCID₅₀ and the rest are vaccinated with the antigen level of approximately 1×10⁵ TCID₅₀. Groups 2, 3 and 5 are vaccinated with a vaccine blended with 10 μg of IL-12 per dose. Group 4 is vaccinated with a vaccine containing BCG as adjuvant. Two doses of each vaccine are given at 20 weeks of age and 23 weeks of age, respectively. To demonstrate the possibility of cross-protection, groups 5 and 6 are heterogeneously challenged with a Broadford strain of E. canis and others are homogeneously challenged with an Ebony strain of E. canis.

Shown in the below Table 4, two out of 5 dogs (40%) in group 3 and two out of 6 dogs (33%) in group 4 are free of thrombocytopenia when the vaccinates are homogeneously challenged with the Ebony strain of E. canis. One hundred percent of the controls (group 1) and the dogs vaccinated with lower dose vaccine (group 2) have severe thrombocytopenia until the study ends. In terms of mortality, five out of 6 (83%) controls are dead or euthanized during the period of observation. The dogs vaccinated with IL-12 adjuvanted lower dose vaccine (group 2) and dogs vaccinated with vaccine adjuvanted with BCG (group 4) have 33% mortality rate. Based on the morbidity and mortality data, the IL-12 adjuvanted E. canis vaccine has significantly enhanced protective immunity against homogeneous E. canis challenge.

As compared with the controls, the addition of IL-12 in combination with EMA-31® and Neocryl® greatly increases the efficacy of E. canis monovalent vaccine and significantly reduces the mortality. The protection induced by the IL-12 combination as shown in groups 2 and 3 is antigen dose-dependent. Further, as compared to BCG, the adjuvant responses induced by the IL-12 combination play a critical role in the reduction of the vaccinated dogs from lethal challenge of E. canis.

Table 4 below shows the results of the pre-immunogenicity study of monovalent E. canis vaccine adjuvanted with recombinant human IL-12.

TABLE 4
Pre-Immunogenicity Study

| Group | Number of | | Challenge | | Mortality | Rectal |
|---|---|---|---|---|---|---|
| Number | Animals | Treatment | Material | Thrombocytopenia | (%) | Temperature |
| 1 | 6 | Control | Ebony | 6/6¹ | 5/6, 83% | 6/6² |
| 2 | 6 | 10e4 EB/IL12 | Ebony | 6/6 | 2/6, 33% | 6/6 |
| 3 | 5 | 10e5 EB/IL12 | Ebony | 3/5 | 0/5, 0% | 3/5 |
| 4 | 6 | 10e5 EB/BCG | Ebony | 4/6 | 2/6, 33% | 6/6 |
| 5 | 6 | 10e5 EB/IL12 | Broadfoot | 2/6 | 0/6, 0% | 2/6 |
| 6 | 6 | Control | Broadfoot | 2/6 | 0/6, 0% | 2/6 |

¹The ratio represents the number of thrombocytopenic dogs per total dogs tested.
²The ratio represents the number of dogs which have elevated rectal temperature per total of dogs in that group.

### EXAMPLE 4

### Evaluation of Humoral Immune Response to Dog Vaccine

A study is performed to determine the effect of a certain adjuvant on the immunogenicity of a modified live and killed viruses and killed bacterin of Canine Duramune® 10 Vaccine (composed of lyophilized live, attenuated canine parvovirus (CPV), canine parainfluenza virus (CPI), canine adenovirus type II (CAV-2), canine adenovirus (CDV) and a diluent containing canine coronavirus (CCV), Leptospira icteroheniorrhagiae (LI), Leptospira canicola (LC), Leptospira grippotyphosa (LG) and Leptospira pomona (LP), killed virus and bacterin fractions, commercially available from Fort Dodge Animal Health, Inc., a veterinary division of Wyeth, Madison, N.J.). To prepare the adjuvant, stock solutions of recombinant human IL-12 (4.45 mg/mL), Duramune® 10 immunogenicity vaccine, EMA-31®, Neocryl® A640 and thimerosal (2% v/v) are used.

For preparation of the test vaccine, the initial adjuvant is prepared by blending Neocryl® and EMA-31® to a final concentration of about 3% and about 1%, respectively. Thimerosal is added at the concentration of about 1:20,000 as preservative.

To prepare the IL-12 adjuvanted diluent, the diluent portion of the Duramune® 10 vaccine is first blended with the above initial adjuvant at a ratio of about 1:10, one part of Duramune® 10 diluent and 9 parts of the initial adjuvant comprising Neocryl® and EMA-31®. The recombinant human IL-12 is then added at a final concentration of about 10 μg or 40 μg per dose.

Prior to use, one part of the lyophilized portion of the Duramune® 10 Vaccine is rehydrated in 9 parts of the IL-12 adjuvanted diluent. Therefore, the fractions of the Duramune® 10 vaccine used in this study is about 10-fold less than the conventional immunogenicity vaccine. In other words, the vaccine tested in this study contains an insufficient amount of antigen (subpotent) as compared to the regular vaccines designed for commercial sale.

A total of 15 dogs are randomized into three groups of 5 dogs each and vaccinated twice subcutaneously at 10 weeks of age and 13 weeks of age. The first group is vaccinated with a vaccine containing about 10 μg of IL-12. The second group is vaccinated with a vaccine containing about 40 μg of IL-12. The third group is vaccinated with a 1:10 diluted Duramune® 10 placebo without IL-12.

The dogs are bled for serum at 0 day post vaccination one (0 DPV1) and 0 day post vaccination two (0 DPV2), 7, 14, 21 and 28 DPV2. The antibody titers for the leptospiras are determined by microagglutination test (MAT).

The results are detailed in the below Tables 5-8. Significant difference between the principal group and the placebo is observed in LP and LG fractions. For LP fraction listed in Table 5, significant difference is observed at 0 DPV2 (group vaccinated with about 10 μg of IL-12) and 21 DPV2 (same group). For the fraction of LG listed in Table 6, the significant difference is observed at 0 DPV2 (group vaccinated with about 40 μg of IL-12), 7, 14 and 21 DPV2 (both 10 and 40 μg groups). No significant difference is observed in the other two fractions (Tables 7 and 8). Therefore, IL-12 addition to the mixture of EMA-31® and Neocryl® is shown to enhance the humoral immune responses to the two leptospiras, LP and LG.

TABLE 5
Results of L. Pomona MAT Assay

| Group | 0 DPV1 | 0 DPV2 | 7 DPV2 | 14 DPV2 | 21 DPV2 |
|---|---|---|---|---|---|
| Duramune ® 10 + | ≦4 | 147¹ | 512 | 388 | 256¹ |
| IL-12 (10 μg) | | | | | |
| Duramune ® 10 + | ≦4 | 128 | 638 | 388 | 194 |
| IL-12 (40 μg) | | | | | |
| Placebo | ≦4 | 74 | 194 | 128 | 49 |
| Environmental | ≦4 | ≦4 | ≦4 | ≦4 | ≦4 |
| Control | | | | | |

¹Number is significant when compared with placebo group.

TABLE 6
Results of L. Grippo MAT Assay

| Group | 0 DPV1 | 0 DPV2 | 7 DPV2 | 14 DPV2 | 21 DPV2 |
|---|---|---|---|---|---|
| Duramune ® 10 + | ≦4 | 11 | 2551² | 2353² | 1176² |
| IL-12 (10 μg) | | | | | |
| Duramune ® 10 + | ≦4 | 37² | 2931² | 2274² | 1084² |
| IL-12 (40 μg) | | | | | |
| Placebo¹ | ≦4 | 6 | 215 | 337 | 215 |
| Environmental | ≦4 | ≦4 | ≦4 | 6 | 8 |
| Control | | | | | |

¹The titer of one dog is 1024 at 0 DPV1 and is excluded from the analysis.
²Number is significant when compared with placebo group

TABLE 7
Results of L. Ictero MAT Assay

| Group | 0 DPV1 | 0 DPV2 | 7 DPV2 | 14 DPV2 | 21 DPV2 | 28 DPV2 |
|---|---|---|---|---|---|---|
| Duramune ® 10 + IL-12 | ≦4 | 8 | 49 | 60 | 32 | 24 |
| (10 μg) | | | | | | |
| Duramune ® 10 + IL-12 | ≦4 | 14 | 105 | 86 | 42 | 32 |
| (40 μg) | | | | | | |
| Placebo | ≦4 | 5 | 74 | 62 | 28 | 21 |
| Environmental Control | ≦4 | ≦4 | ≦4 | ≦4 | ≦4 | ≦4 |

TABLE 8
Results of L. Canicola MAT Assay

| Group | 0 DPV1 | 0 DPV2 | 7 DPV2 | 14 DPV2 | 21 DPV2 |
|---|---|---|---|---|---|
| Duramune ® 10 + | ≦4 | 6 | 256 | 194 | 64 |
| IL-12 (10 μg) | | | | | |
| Duramune ® 10 + | ≦4 | 16 | 338 | 278 | 223 |
| IL-12 (40 μg) | | | | | |
| Placebo¹ | ≦4 | 5 | 159 | 139 | 49 |
| Environmental | ≦4 | ≦4 | ≦4 | ≦4 | ≦4 |
| Control | | | | | |

¹The titer of one dog is 1024 at 0 DPV1 and is excluded from the analysis.
²Number is significant when compared with placebo group

### EXAMPLE 5

### Effect of Adjuvant of Immunogenicity of Cat Vaccine

To determine whether recombinant human IL-12 can enhance the immunogenicity of an FIV-FeLV vaccine, IL-12 is blended with inactivated feline immunodeficiency virus (FIV) and feline leukemia virus (FeLV) at 5 μg per dose after EMA-31®, Neocryl® A640 and MVP® (an oil-in-water emulsion of light mineral oil, commercially available from Modern Veterinary Products, Omaha, Nebr.) are added to the vaccine. The challenge route of administration for the vaccine is intraperitoneally. One group of 20 kittens, eight weeks of age, are vaccinated twice with the FIV-FeLV vaccine, another group of 20 age-matched kittens are vaccinated with the same vaccine supplemented with IL-12. Three weeks following the completion of vaccination, all vaccinates are challenged with virulent FeLV along with nine age-matched controls. The challenged cats are monitored weekly for viremia for 15 weeks. To monitor the challenged cats for FeLV viremia, the serum samples are tested weekly for the presence of FeLV p27 antigen using IDEXX FeLV antigen test kit. A cat is considered persistently infected with FeLV when FeLV p27 is detected on three consecutive sampling points during weeks 3 through 12 after challenge exposure. All nine controls are found to become persistently infected with FeLV. Five out of 20 vaccinates which receive the FIV-FeLV vaccine are found to become persistently infected with FeLV while only one out of 20 vaccinates which receive the FIV-FeLV vaccine supplemented with IL-12 are found to become persistently infected with FeLV. This result indicates that IL-12 in combination with EMA-31®, Neocryl® and MVPO may be used to enhance the immunogenicity of FeLV vaccines.

In the foregoing, there has been provided a detailed description of particular embodiments of the present invention for the purpose of illustration and not limitation. It is to be understood that all other modifications, ramifications and equivalents obvious to those having skill in the art based on this disclosure are intended to be included within the scope of the invention as claimed.

I claim:

1. A composition for enhancing the immunogenicity of a veterinary vaccine which comprises a pharmacologically effective amount of an immunomodulator and an immunoadjuvant.

2. The immunogenicity enhancing composition according to claim 1, wherein the immunomodulator is selected from the group consisting of a cytokine, an interferon, tumor necrosis factor, transforming growth factor, colony stimulating factor and a combination thereof.

3. The immunogenicity enhancing composition according to claim 2, wherein the immunomodulator is a cytokine.

4. The immunogenicity enhancing composition according to claim 3, wherein the immunomodulator is interleukin-12.

5. The immunogenicity enhancing composition according to claim 4, wherein the immunomodulator is a homologous animal, recombinant human or recombinant murine interleukin-12.

6. The immunogenicity enhancing composition according to claim 5, wherein the immunoadjuvant is selected from the group consisting of a saponin, a metabolizable oil, a block copolymer, an ethylene/maleic copolymer, an acrylic acid copolymer, an acrylic acid copolymer emulsion, a mineral oil emulsion and a mixture thereof.

7. The immunogenicity enhancing composition according to claim 6, wherein the immunoadjuvant is saponin.

8. The immunogenicity enhancing composition according to claim 6, wherein the metabolizable oil is squalene or squalane.

9. The immunogenicity enhancing composition according to claim 8, wherein the metabolizable oil is squalane.

10. The immunogenicity enhancing composition according to claim 6, wherein the block copolymer is a polyoxypropylene-polyoxyethylene block copolymer.

11. The immunogenicity enhancing composition according to claim 6, wherein the ethylene/maleic copolymer is a linear ethylene/maleic anhydride copolymer having approximately equal amounts of ethylene and maleic anhydride and an estimated average molecular weight of about 75,000 to 100,000.

12. The immunogenicity enhancing composition according to claim 6, wherein the acrylic acid copolymer is a mixture of styrene and an uncoalesced aqueous acrylic acid copolymer of acrylic acid and methacrylic acid.

13. The immunogenicity enhancing composition according to claim 12, wherein the mixture is emulsified.

14. The immunogenicity enhancing composition according to claim 6, wherein the mineral oil emulsion is an oil-in-water emulsion of light mineral oil.

15. The immunogenicity enhancing composition according to claim 6, wherein the immunoadjuvant is a mixture of a polyoxypropylene-polyoxyethylene block copolymer and squalane.

16. The immunogenicity enhancing composition according to claim 6, wherein the immunoadjuvant is a mixture of a linear ethylene/maleic anhydride copolymer, having approximately equal amounts of ethylene and maleic anhydride and an estimated average molecular weight of about 75,000 to 100,000, and an acrylic acid copolymer emulsion comprising an emulsified mixture of styrene and an uncoalesced aqueous acrylic acid copolymer of acrylic acid and methacrylic acid.

17. The immunogenicity enhancing composition according to claim 6, wherein the immunoadjuvant is a mixture of a linear ethylene/maleic anhydride copolymer, having approximately equal amounts of ethylene and maleic anhydride and an estimated average molecular weight of about 75,000 to 100,000, an acrylic acid copolymer emulsion comprising an emulsified mixture of styrene and an uncoalesced aqueous acrylic acid copolymer of acrylic acid and methacrylic acid, and a mineral oil emulsion.

18. An improved veterinary vaccine composition which comprises an effective immunizing amount of an antigen, an immunomodulator, an immunoadjuvant and a pharmaceutically acceptable carrier.

19. The vaccine composition according to claim 18, wherein the immunomodulator is selected from the group consisting of a cytokine, an interferon, tumor necrosis factor, transforming growth factor, colony stimulating factor and a combination thereof.

20. The vaccine composition according to claim 19, wherein the immunomodulator is a cytokine.

21. The vaccine composition according to claim 20, wherein the immunomodulator is interleukin-12.

22. The vaccine composition according to claim 21, wherein the immunomodulator is a homologous animal, recombinant human or recombinant murine interleukin-12.

23. The vaccine composition according to claim 22, wherein the immunoadjuvant is selected from the group consisting of a saponin, a metabolizable oil, a block copolymer, an ethylene/maleic copolymer, an acrylic acid copolymer, an acrylic acid copolymer emulsion, a mineral oil emulsion and a mixture thereof.

24. The vaccine composition according to claim 23, wherein the immunoadjuvant is saponin.

25. The vaccine composition according to claim 23, wherein the metabolizable oil is squalene or squalane.

26. The vaccine composition according to claim 25, wherein the metabolizable oil is squalane.

27. The vaccine composition according to claim 23, wherein the block copolymer is a polyoxypropylene-polyoxyethylene block copolymer.

28. The vaccine composition according to claim 23, wherein the ethylene/maleic copolymer is a linear ethylene/maleic anhydride copolymer having approximately equal amounts of ethylene and maleic anhydride and an estimated average molecular weight of about 75,000 to 100,000.

29. The vaccine composition according to claim 23, wherein the acrylic acid copolymer is a mixture of styrene and an uncoalesced aqueous acrylic acid copolymer of acrylic acid and methacrylic acid.

30. The vaccine composition according to claim 29, wherein the mixture is emulsified.

31. The vaccine composition according to claim 23, wherein the mineral oil emulsion is an oil-in-water emulsion of light mineral oil.

32. The vaccine composition according to claim 27, wherein the immunoadjuvant is a mixture of the polyoxypropylene-polyoxyethylene block copolymer and a metabolizable oil.

33. The vaccine composition according to claim 32, wherein the metabolizable oil is squalane.

34. The vaccine composition according to claim 23, wherein the immunoadjuvant is a mixture of the ethylene/maleic anhydride copolymer and an acrylic acid copolymer emulsion.

35. The vaccine composition according to claim 34, wherein the immunoadjuvant further comprises a mineral oil emulsion.

36. The vaccine composition according to claim 23, wherein the antigen is selected from the group consisting of Bovine Respiratory Syncytial Virus, herpes simplex virus type 1, bovine virus diarrhea, parainfluenza-3 virus, canine parvovirus, canine parainfluenza virus, canine adenovirus type II, canine adenovirus, canine coronavirus, rabies virus, feline immunodeficiency virus, feline leukemia virus, feline coronavirus, Porcine Reproductive and Respiratory Syndrome (PRRS) Virus, chicken herpes virus, Chlamydia, Ehrlichia, Pasteurella, Haemophilus, Salmonella, Staphylococcus, Streptococcus, Mycoplasma, Borrelia, Leptospira, Coccidia, Hemosporidia, Amoebida, Trypanosoma, Leishmania, Giardia, Histomonas, Coccidioides, Histoplasma, Blastomyces, Aspergillus, Cryptococcus and a combination thereof.

37. The vaccine composition according to claim 36, wherein the antigen is Streptococcus equi and the immunoadjuvant is a saponin.

38. The vaccine composition according to claim 36, wherein the antigen is Bovine Respiratory Syncytial Virus and the immunoadjuvant is a mixture of the block copolymer and the metabolizable oil.

39. The vaccine composition according to claim 36, wherein the antigen is Ehrlichia canis and the immunoadjuvant is a mixture of the ethylene/maleic copolymer and the acrylic acid copolymer emulsion.

40. The vaccine composition according to claim 36, wherein the antigen is a combination of canine parvovirus, canine parainfluenza virus, canine adenovirus type II, canine adenovirus, canine coronavirus, Leptospira icterohemorrhagiae, Leptospira canicola, Leptospira grippotyphosa and Leptospira pomona, and the immunoadjuvant is a mixture of the ethylene/maleic copolymer and the acrylic acid copolymer emulsion.

41. The vaccine composition according to claim 36, wherein the antigen is a combination of feline immunodeficiency virus and feline leukemia virus, and the immunoadjuvant is a mixture of the ethylene/maleic copolymer, the acrylic acid copolymer emulsion and the mineral oil emulsion.

42. The vaccine composition according to claim 18, further comprising a preservative.

43. The vaccine composition according to claim 18, further comprising a secondary adjuvant.

44. The vaccine composition according to claim 43, wherein the secondary adjuvant is selected from the group consisting of a stabilizer, an emulsifier, aluminum hydroxide, aluminum phosphate, a pH adjuster, a surfactant, a liposome, an iscom adjuvant, a synthetic glycopeptide, an extender, carboxypolymethylene, bacterial cell wall, a derivative of a bacterial cell wall, vaccinia, an animal poxvirus protein, a subviral particle adjuvant, cholera toxin, N,N-dioctadecyl-N′,N′-bis(2-hydroxyethyl)propanediamine, monophosphoryl lipid A, dimethyldioctadecyl-ammonium bromide and a mixture thereof.

45. A method for potentiating, accelerating or extending the immunogenicity of a weak, immunosuppressive or marginally safe antigen which comprises administering to an avian or mammalian species a pharmacologically effective amount of the immunogenicity enhancing composition according to claim 1 before, concurrently with, sequentially with or after the administration of the weak, immunosuppressive or marginally safe antigen.

46. A method for potentiating, accelerating or extending the immunogenicity of a weak, immunosuppressive or marginally safe antigen which comprises administering to an avian or mammalian species an effective immunizing amount of the vaccine composition according to claim 18.

47. The method according to claim 45 or 46, which comprises administering the immunogenicity enhancing or vaccine composition subcutaneously, intramuscularly, intradermally, intraperitoneally, intranasally, intrabuccally, transdermally or orally.

48. The method according to claim 45 or 46, which comprises administering the immunogenicity enhancing or vaccine composition which contains an immunomodulator selected from the group consisting of a cytokine, an interferon, tumor necrosis factor, transforming growth factor, colony stimulating factor and a combination thereof.

49. The method according to claim 48, which comprises administering the immunogenicity enhancing or vaccine composition which contains the cytokine comprising a homologous animal, recombinant human or recombinant murine interleukin-12.

50. The method according to claim 49, which comprises administering the immunogenicity enhancing or vaccine composition which contains the immunoadjuvant selected from the group consisting of a saponin, a metabolizable oil, a block copolymer, an ethylene/maleic copolymer, an acrylic acid copolymer, an acrylic acid copolymer emulsion, a mineral oil emulsion and a mixture thereof.

51. The method according to claim 50, which comprises administering the vaccine composition which contains an antigen selected from the group consisting of Bovine Respiratory Syncytial Virus, herpes simplex virus type 1, bovine virus diarrhea, parainfluenza-3 virus, canine parvovirus, canine parainfluenza virus, canine adenovirus type II, canine adenovirus, canine coronavirus, rabies virus, feline immunodeficiency virus, feline leukemia virus, feline coronavirus, Porcine Reproductive and Respiratory Syndrome (PRRS) Virus, chicken herpes virus, Chlamydia, Ehrlichia, Pasteurella, Haemophilus, Salmonella, Staphylococcus, Streptococcus, Mycoplasma, Borrelia, Leptospira, Coccidia, Hemosporidia, Amoebida, Trypanosoma, Leishmania, Giardia, Histomonas, Coccidioides, Histoplasma, Blastomyces, Aspergillus, Cryptococcus and a combination thereof.
